(19) United States
(12) Patent Application Publication
(10) Pub. No.: US 2014/0370077 A1
(43) Pub. Date: Dec. 18, 2014

(54) TRANSDERMAL DRUG DELIVERY SYSTEM CONTAINING FENTANYL

(71) Applicant: NAL PHARMACEUTICALS, LTD., Causeway Bay (HK)

(72) Inventors: Hoo-Kyun Choi, Gwangju (KR); Je Phil Ryoo, Princeton, NJ (US)

(21) Appl. No.: 14/207,462

(22) Filed: Mar. 12, 2014

Related U.S. Application Data
(60) Provisional application No. 61/780,107, filed on Mar. 13, 2013.

(51) Int. Cl. A61K 9/70; A61K 31/4468

(52) U.S. Cl. CPC A61K 9/7061; A61K 31/4468
USPC 424/448; 514/329; 604/307; 156/243

(57) ABSTRACT

The present invention provides a transdermal drug delivery system comprising fentanyl or its pharmaceutically acceptable salt and method of making the same.

### CROSS-REFERENCE TO RELATED APPLICATION

This application claims the benefit of priority from U.S. Provisional Application Ser. No. 61/780,107 filed Mar. 13, 2013, and is a continuation-in-part of PCT/KR2012/005803 filed Jul. 20, 2012, which claims the benefit of priority from U.S. Provisional Patent Application Ser. No. 61/536,141 filed Sep. 19, 2011, the contents of each of which are incorporated herein by reference.

### FIELD OF INVENTION

The present invention relates to a transdermal drug delivery system comprising fentanyl or a pharmaceutically acceptable salt thereof as an active ingredient, more specifically to a transdermal drug delivery system comprising a drug-containing matrix layer the matrix of which is formed with an acrylate-rubber hybrid adhesive having less than 60 μm thickness and low contents of fentanyl.

### BACKGROUND OF INVENTION

Fentanyl is known to have more than about 80 times stronger than morphine, it has been used to reduce pain in patient with cancer of after surgery. Fentanyl citrate is administered intravenous (i.v.) or orally or via buccal or fentanyl base is administered transdermal delivery. However, it is known that above the effective amount of the dose in the plasma can cause side effect such as respiratory depression or muscular rigidity.

The pain in cancer patients or after surgery is not temporary but could be chronic and continuous and repetitive administration of fentanyl via i.v. or buccal can cause discomfort for the patient and may even cause reduce efficacy in treating pain. Injection i.v. can even cause severe side effects.

In contrary, transdermal delivery is desirable option as it can provide a sustained delivery of fentanyl by one time application for even several days. In addition, as the concentration of fentanyl in plasma can be maintained constant to reduce or prevent side effect often caused by i.v. injection, which can cause initial high concentration of fentanyl in plasma to result in the side effects. Furthermore, transdermal delivery is easy to apply and easy to remove to provide an advantage in any emergency situation.

Most transdermal patch containing fentanyl employs silicon adhesive. For example, U.S. Pat. No. 4,588,580 teaches a matrix type transdermal patch where fentanyl is contained in silicon or polyisobutylene class adhesives and U.S. Pat. No. 5,186,939 teaches a transdermal patch comprising fentanyl dissolved in an amine-resistant polydimethylsiloxane. However, fentanyl has a low solubility in silicon or polyisobutylene class adhesives and high contents of fentanyl in a high concentration in these adhesives can result in a crystallization of fentanyl and eventually low permeation of fentanyl due to the crystallized fentanyl in the patch. Also, an adhesive like polyisobutylene does not have enough adhesive property to properly support the transdermal patch long enough to deliver the drug for a long period of time.

Furthermore, U.S. Patent Application Publication 2009/0238861 teaches a transdermal patch where fentanyl is suspended in solvated silicone adhesives. However, it is not easy to suspend the solid particles evenly through the matrix, manufacturing could be a problematic. As the permeation rate depends on the particle size, permeation of fentanyl may also experience difficulties. In addition, Korean Patent 10-0895188 introduced a micro reservoir within the matrix layer to prevent overdose of fentanyl. However, an additional process to prepare the micro reservoir can increase the production cost significantly.

U.S. Patent Application Publication 2011/0111013 suggests suspending the drug in a gel to resolve the problem of inconsistent solubility, irregular permeation and other problems caused by polyisobutylene class adhesives. However, the patch still contains undissolved drug and could not resolve all previous problems.

U.S. Patent Application Publication 2011/0038918 and Korean Patent 10-0904158 teach fentanyl containing transdermal patch using acrylate adhesives. Korean Patent Application Publication 10-2009-0101579, filed by the present inventors, also teaches a fentanyl containing transdermal patch having acrylate adhesives and, optionally, an absorption enhancer such as polyethylene glycol-12 palm kernel glyceride. Even though fentanyl's solubility in acrylate adhesives is high, permeation of fentanyl was not satisfactory and requires a high contents of fentanyl in the transdermal patch to result in wasting the expensive fentanyl and it is very likely the undelivered fentanyl will remain in the patch.

### SUMMARY OF INVENTION

The present invention provides a fentanyl containing transdermal drug delivery system having improved skin permeation and absorption which allows low contents of fentanyl in the transdermal drug delivery system and also with reduced cost of manufacturing by having simpler process for preparation.

The present invention provide a fentanyl containing transdermal drug delivery system having certain adhesives such as acryl-rubger hybrid adhesives in a fentanyl containing matrix, which can also provide improved skin permeation and absorption to allow low contents of fentanyl in the system but to deliver fentanyl longer time period.

Preferably the transdermal drug delivery system of the present invention has less than 60 μm thickness, more preferably less than 50 μm thickness, even more preferably 40 μm thickness.

The transdermal drug delivery system of the present invention not only show high skin penetration rate but also continuously maintain a therapeutically effective blood concentration for at least 24 hours. And also, the present invention provides a transdermal drug delivery system, which can inhibit recrystallization of fentanyl and maintain skin penetration rate intact, even during the long-term storage.

### BRIEF DESCRIPTION OF DRAWINGS

FIG. 1 shows the results obtained by measuring skin penetration rates of the transdermal drug delivery systems according to the Examples prepared.

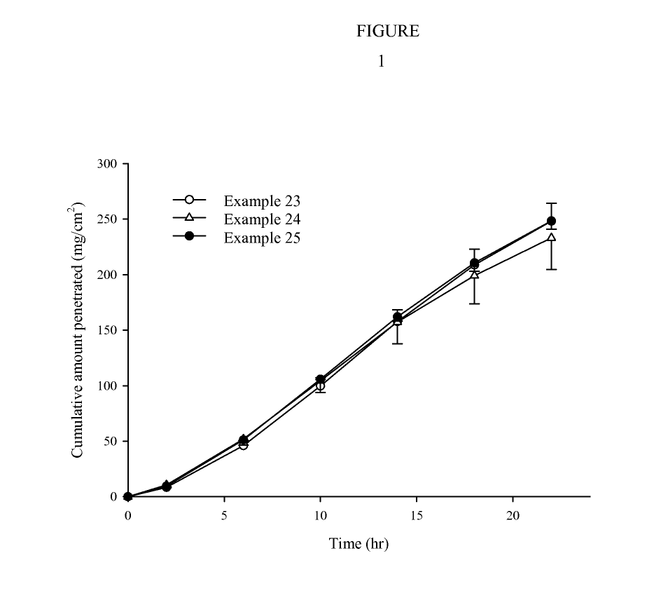

### DETAILED DESCRIPTION OF INVENTION

Alternatively, the present invention provides a transdermal drug delivery system containing back support or back film, drug-containing matrix containing fentanyl or pharmaceutical as the active ingredient and acryl-rubber hybrid as the adhesive.

That is, the present invention provides a fentanyl-containing transdermal drug delivery system, both showing high skin penetration rate continuously for more than 24 hours and having an excellent stability.

In accordance with an aspect of the present invention, there is provided a transdermal drug delivery system comprising a drug-containing matrix layer comprising: (a) fentanyl or a pharmaceutically acceptable salt thereof as an active ingredient; and (b) an acrylate-rubber hybrid as an adhesive. In an embodiment of the present invention, the transdermal drug delivery system may consist of a backing layer, the drug-containing matrix layer, and a release layer.

The acrylate-rubber hybrid may be an acrylic polymer comprising a C₄-C₁₈ alkyl acrylate monomer grafted with ethylene-butylene macromers. The C₄-C₁₈ alkyl acrylate monomers may contain monomers having a glass transition temperature of not more than −30° C. More specifically, C₄-C₁₈ alkyl acrylate monomers is one or more selected from among butyl acrylate, amyl acrylate, hexyl acrylate, 2-ethylhexyl acrylate, isooctyl acrylate, decyl acrylate, or dodecyl acrylate, preferably 2-ethylhexyl acrylate.

In the transdermal drug delivery system according to the present invention, the fentanyl or its pharmaceutically acceptable salt may be present in an amount ranging from 5 to 40% by weight, based on the total weight of the drug-containing matrix layer and the acrylate-rubber hybrid adhesive may be present in an amount ranging from 60 to 95% by weight, based on the total weight of the drug-containing matrix layer.

The transdermal drug delivery system according to the present invention may further comprise an acrylate polymer or a methacrylate polymer as a crystallization-inhibiting agent. The crystallization-inhibiting agent may be present in an amount ranging from 1 to 10% by weight, based on the total weight of the drug-containing matrix layer. The crystallization-inhibiting agent may be a copolymer of butyl methacrylate, 2-dimethylaminoethyl methacrylate, and methyl methacrylate in a weight ratio of 1:2:1.

The transdermal drug delivery system according to the present invention may further comprise one or more absorption enhancers selected from the group consisting of terpenes; non-ionic surfactants; polyoxyethylene alkyl ethers; polyethylene glycol glycerides; fatty alcohols; fatty acid esters; propylene glycol esters; polyglyceryl fatty acid esters; sugar esters; fatty acids; glycerols; alkyl 2-ethyl hexanates; and diethoxylethyl succinates. The absorption enhancer may be present in an amount ranging from 1 to 20% by weight, based on the total weight of the drug-containing matrix layer.

Preferably, the absorption enhancer may be one or more selected from the group consisting of polyethylene glycol palm kernel glyceride, polyoxyethylene lauryl ether, polyglyceryl-3 oleate, lauryl alcohol, and oleyl alcohol.

The transdermal drug delivery system according to the present invention comprises a matrix obtained by using an acrylate-rubber hybrid as an adhesive, which can increase the diffusion rate of fentanyl from the matrix layer. Therefore, the transdermal drug delivery system according to the present invention can not only show high skin penetration rate but also continuously maintain a therapeutically effective blood concentration for at least 24 hours. And also, the transdermal drug delivery system of the present invention can inhibit recrystallization of fentanyl and maintain skin penetration rate intact, even during the long-term storage. Therefore, the transdermal drug delivery system according to the present invention can improve drug compliance of patients suffering from pain.

Furthermore, the present invention can reduce the thickness of the drug-containing matrix significantly, preferably less than 60 μm and achieve high absorption and permeation through the skin consistently even at the lower contents or concentration of the drug per unit area. The low contents of drug per unit area also will reduce the remaining drugs in the matrix after the treatment to reduce the waste of the drug, which can also provide benefit of reduction of drug abuse or overdose.

The transdermal drug delivery system according to the present invention comprises a matrix layer containing fentanyl or pharmaceutically effective salts thereof, which is evenly well distributed throughout the matrix layer to provide consistent delivery of fentanyl.

As used herein, the term “acrylate-rubber hybrid” adhesive refers to an acrylic polymers containing acrylate monomers grafted with a rubber macromere such as ethylene-butylene macromers. The above-mentioned ‘grafting’ or ‘hybrid’ means the above macromers are attached or conjugated at the functional groups of the alkyl acrylate monomers. The ‘grafting’ is accomplished by dissolving alkyl acrylate or rubber macromers in ethyl acetate, hexane or mixture of both and adding an initiator such as, but not limited to, azobis(isobutyronitrile, AIBN). More detailed ‘grafting’ procedure is disclosed in U.S. Pat. No. 6,670,417, the contents of each of which are incorporated herein by reference.

The acrylate-rubber hybrid adhesive in the present application is, preferably, grafted acryl polymers containing C₄-C₁₈ alkyl acrylate monomers grafted with ethylene-butylene macromers or rubber macromere, wherein the C₄-C₁₈ alkyl acrylate monomers have a glass transition temperature of not more than −30° C. For example, the C₄-C₁₈ alkyl acrylate monomers may be one of more selected from among butyl acrylate, amyl acrylate, hexyl acrylate, 2-ethylhexyl acrylate, isooctyl acrylate, decyl acrylate, or dodecyl acrylate and preferably 2-ethylhexyl acrylate. More preferably, the acrylate-rubber hybrid adhesive may be one or more selected from commercially available acrylate-rubber hybrids, i.e., Duro-Tak™ 87-502A (National Starch), Duro-Tak™ 87-502B (National Starch), Duro-Tak™ 87-503A (National Starch), Duro-Tak™ 87-504A (National Starch), or Duro-Tak™ 87-504B (National Starch).

More preferably, the acrylate-rubber hybrid adhesives may be one of more selected from commercially available acrylate-rubber hybrids, Duro-Tak™ 87-502B (National Starch), and Duro-Tak™ 87-504B (National Starch). Also, it can be further distinguished that different acrylate-rubber hybrid adhesives can be prepared by different of solvent systems. The final composition of hybrid adhesives can be quite different by the solvent used in the process. Therefore, even though the individual monomers of polymer employed for hybrid may be same but the chemical composition or structure of the final hybrid adhesive could be different to provide different properties.

The hybrid adhesives act as a solvent to dissolve the drug substance in the preparation of the transdermal patch. Thus, different development approach could be required for using the different hybrid adhesives. The formulation for developing a transdermal patch should be modified significantly according to the hybrid adhesive or solvent compositions. Since their physical properties and the compatibility of adhesives to drug substance were changed, the formulation development of patch should be approached with totally different methods to maintain the better stability of the final formula.

The present invention provides a transdermal drug delivery system, which comprises a drug-containing matrix layer comprising: (a) fentanyl or a pharmaceutically acceptable salt thereof as an active ingredient; and (b) an acrylate-rubber hybrid as an adhesive.

In an embodiment of the present invention, the transdermal drug delivery system may consist of a backing layer, the drug-containing matrix layer, and a release layer.

In the transdermal drug delivery system according to the present invention, the acrylate-rubber hybrid is used as an adhesive; and the acrylate-rubber hybrid adhesive forms a matrix in the drug-containing matrix layer. That is, fentanyl or its pharmaceutically acceptable salt is homogeneously dispersed in the acrylate-rubber hybrid adhesive, thereby forming the drug-containing matrix layer.

It is newly found by the present invention that a matrix formed from the acrylate-rubber hybrid having low glass transition temperature can improve flexibility of polymer chains, thereby increasing a diffusion rate of an active ingredient (i.e., fentanyl or its pharmaceutically acceptable salt) to the skin from the matrix layer. Therefore, the use of the acrylate-rubber hybrid leads to higher skin penetration rate and excellent adhesive force, in comparison with not only acrylic adhesives having no functional group (for example, Duro-Tak™ 87-4098, Duro-Tak™ 87-900A, Duro-Tak™ 87-9301 etc.) but also other acrylic adhesives having hydroxyl or carboxyl functional group (for example, Duro-Tak™ 87-2516, Duro-Tak™ 87-2510, Duro-Tak™ 87-2525, Duro-Tak™ 87-2596, Duro-Tak™ 87-2825, Duro-Tak™ 87-2502, Duro-Tak™ 87-2979, Duro-Tak™ 87-2074 etc.).

The acrylate-rubber hybrid adhesive may be used in an amount sufficient to form a matrix layer, for example, in an amount ranging from 60 to 95% by weight, based on the total weight of the drug-containing matrix layer.

In the transdermal drug delivery system according to the present invention, the fentanyl or its pharmaceutically acceptable salt may be used in an amount sufficient to obtain a therapeutically effective blood concentration, for example, in an amount ranging from 5 to 40% by weight, preferably from 5 to 20% by weight, more preferably 7-16% by weight based on the total weight of the drug-containing matrix layer. If the amount of fentanyl or its pharmaceutically acceptable salt is more than 40% by weight, drug crystals may be formed in the transdermal drug delivery system, which results in reducing adhesive force or lowering absorption rate of the drug.

In the transdermal drug delivery system according to the present invention, fentanyl does not crystallize out of the system even after duration of time to maintain thermodynamic activity of fentanyl or its pharmaceutically active salts. Therefore, the thickness of the system can be significantly reduced to less than 60 μm, preferably less than 50 μm, more preferably less than 40 μm, or even more preferably between 15-40 μm considering the adhesiveness and duration of application. In other words, reduction of the thickness of the matrix later and high concentration of fentanyl in a unit area but still provides consistently high permeation of fentanyl.

The transdermal drug delivery system according to the present invention may further comprise a crystallization-inhibiting agent. The crystallization-inhibiting agent may be an acrylate polymer or a methacrylate polymer, preferably a copolymer of butyl methacrylate, 2-dimethylaminoethyl methacrylate, and methyl methacrylate in a weight ratio of 1:2:1 (for example, Eudragit™ E100). The crystallization-inhibiting agent may be present in an amount ranging from 1 to 10% by weight, based on the total weight of the drug-containing matrix layer.

Alternatively, the transdermal drug delivery system according to the present invention does not contain crystallization-inhibiting agent.

And also, the transdermal drug delivery system according to the present invention may comprise a conventional absorption enhancer used in the field of a transdermal drug delivery system. The absorption enhancer may be present in an amount ranging from 1 to 20% by weight, preferably from 5 to 15% by weight, based on the total weight of the drug-containing matrix layer. If the amount of an absorption enhancer is more than 20% by weight, adhesive force may be reduced; or cold flow may occur due to weaken cohesive force.

The absorption enhancer may be one or more selected from the group consisting of terpenes; surfactants; polyoxyethylene alkyl ethers; fatty alcohols; sugar esters; glycerols; alkyl 2-ethyl hexanates; and diethoxylethyl succinates.

Examples of the terpenes include cineole, limonene, etc.

Examples of the surfactants include isopropyl myristate, isopropyl palmitate, 2-(2-ethoxyethoxy) ethanol, oleic acid oleyl ester, caprylocaproyl macrogolglyceride, oleoyl macrogolglyceride, diisopropyl dirrerate, diisopropyl adipate, hexyl laurate, polysorbate, sorbitan oleate, etc.

Examples of the polyoxyethylene alkyl ethers include polyethylene glycol palm kernel glyceride, 2-ethyl hexyl hydroxystearate, polyoxyethylene lauryl ether, polyoxyethylene cetyl ether, etc.

Examples of the fatty alcohols include polyglyceryl-3 oleate, polyethylene glycol almond glyceride, lauryl alcohol, oleyl alcohol, etc.

Examples of the sugar esters include sucrose stearate, sucrose palmitate, sucrose laurate, sucrose behenate, sucrose oleate, sucrose erucate, etc.

Examples of the alkyl 2-ethyl hexanates include 2-ethylhexanonate, cetyl 2-ethylhexanonate, stearyl 2-ethylhexanonate, etc.

Among the above mentioned absorption enhancers, the polyoxyethylene alkyl ethers and/or the fatty alcohols may be preferably used. More preferably, the absorption enhancer may be one or more selected from the group consisting of polyethylene glycol palm kernel glyceride (for example, Crovol™ A40), polyoxyethylene lauryl ether (for example, Brij™ 30, Brij™ 52, etc.), polyglyceryl-3 oleate (for example, Plurol Oleique™ cc497), lauryl alcohol, and oleyl alcohol. Most preferably, polyoxyethylene lauryl ether (for example, Brij™ 30) may be used as an absorption enhancer.

The transdermal drug delivery system of the present invention may be prepared by forming the drug-containing matrix layer on a release layer and then forming a backing layer thereon. For the release layer, conventional release liners or their laminates used in the field of a transdermal drug delivery system may be used. For example, there may be used a film, a paper, or a laminates thereof, which made of polyethylene, polyester, polyvinyl chloride, polyvinylidene chloride, etc. coated with silicone resin or fluoride resin. And also, drug non-absorbable and flexible materials conventionally used in the field of a transdermal drug delivery system may be used as the backing layer (also referred to as “backing membrane”). For example, there may be used polyolefin, polyether, a multi-layer ethylene vinyl acetate film, polyester, polyurethane, etc. The transdermal drug delivery system of the present invention may be prepared, for example by dissolving fentanyl or its pharmaceutically acceptable salt and an acrylate-rubber hybrid adhesive, optionally along with an absorption enhancer and/or a crystallization-inhibiting agent, in an appropriate solvent (e.g., ethyl acetate, etc.), casting the resulting solution on a release liner coated with silicone followed by drying the mixture, and then laminating a backing layer.

The present invention will be described in further detail with reference to the following examples and experimental examples. These examples and experimental examples are for illustrative purposes only and are not intended to limit the scope of the present invention.

### EXAMPLES

### Examples 1 to 31

The transdermal drug delivery systems were prepared according to the components and amounts shown in Tables 1-5. To a mixture of fentanyl and an acrylate-rubber hybrid adhesive, was added ethyl acetate as a solvent so as to attain to 25% of solid content. After stirring each mixture, the resulting each solution was casted on a release liner coated with silicone, followed by drying the mixture. A polyethylene film (Cotran™ 9720) was laminated onto the resulting each layer to form a backing membrane, so as to prepare each fentanyl-containing transdermal drug delivery system.

TABLE 1

| | | Example (% by weight) | | | | | | | | | | |
|---|---|---|---|---|---|---|---|---|---|---|---|---|
| | Component | 1 | 2 | 3 | 4 | 5 | 6 | 7 | 8 | 9 | 10 | 11 |
| Active | Fentanyl | 11 | 10 | 10 | 10 | 10 | 11 | 12 | 12 | 7.5 | 9 | 13 |
| ingredient | | | | | | | | | | | | |
| Acrylate- | Duro-Tak ™ 87- | 89 | 85 | 88 | 85 | 85 | 81 | 80 | 80 | 88 | 86 | 82 |
| rubber hybrid | 502A | | | | | | | | | | | |
| adhesive | | | | | | | | | | | | |
| Absorption | Brij ™ 30 | | 5 | | | | | | | | | |
| enhancer | Plurololeique ™ | | | 2 | | | | | | | | |
| | CC497 | | | | | | | | | | | |
| | Labrafil ™ | | | | 5 | | | | | | | |
| | M1944CS | | | | | | | | | | | |
| | Lauroglycol ™ | | | | | 5 | 8 | 8 | 8 | 5 | 5 | 5 |
| | FCC | | | | | | | | | | | |

| | | | | | | | | | | | |
|---|---|---|---|---|---|---|---|---|---|---|---|
| Matrix Thickness (μm) | 40 | 40 | 40 | 40 | 40 | 30 | 30 | 35 | 60 | 50 | 30 |

TABLE 2

| | | Example (% by weight) | | | | | | | |
|---|---|---|---|---|---|---|---|---|---|
| | Component | 12 | 13 | 14 | 15 | 16 | 17 | 18 | 19 |
| Active | Fentanyl | 10 | 10 | 11 | 11 | 11 | 11 | 11 | 11 |
| ingredient | | | | | | | | | |
| Acrylate-rubber | Duro-Tak ™ 87- | 85 | | 84 | 84 | 84 | 84 | 84 | 84 |
| hybrid adhesive | 504A | | | | | | | | |
| | Duro-Tak ™ 87- | | 85 | | | | | | |
| | 504B | | | | | | | | |
| Absorption | Lauroglycol ™ | 5 | 5 | 5 | | | | | |
| enhancer | FCC | | | | | | | | |
| | Labrasol ™ | | | | 5 | | | | |
| | Isopropyl | | | | | 5 | | | |
| | palmitate | | | | | | | | |
| | Span 80 ™ | | | | | | 5 | | |
| | Brij ™ 72 | | | | | | | 5 | |
| | Cineole | | | | | | | | 5 |

| | | | | | | | | |
|---|---|---|---|---|---|---|---|---|
| Matrix Thickness (μm) | 45 | 45 | 40 | 40 | 40 | 40 | 40 | 40 |

TABLE 3

| | | Example | | |
|---|---|---|---|---|
| | | (% by weight) | | |
| | Component | 20 | 21 | 22 |
| Active ingredient | Fentanyl | 11 | 11 | 12 |
| Acrylate-rubber hybrid | Duro-Tak ™ 87-504A | 81 | 81 | 84 |
| adhesive | | | | |
| Absorption enhancer | Lauroglycol ™ FCC | 8 | 8 | 4 |

| | | | |
|---|---|---|---|
| Matrix Thickness (μm) | 35 | 40 | 40 |

TABLE 4

| | | Example | | |
|---|---|---|---|---|
| | | (% by weight) | | |
| | Component | 23 | 24 | 25 |
| Active ingredient | Fentanyl | 10 | 10 | 10 |
| Acrylate-rubber hybrid | Duro-Tak ™ 87-502B | 85 | | |
| adhesive | Duro-Tak ™ 87-504B | | 85 | |
| | Duro-Tak ™ 87-504A | | | 85 |
| Absorption enhancer | Lauroglycol ™ | 5 | 5 | 5 |

| | | | |
|---|---|---|---|
| Matrix Thickness (μm) | 40 | 40 | 40 |

TABLE 5

| | | Example (% by weight) | | | | | |
|---|---|---|---|---|---|---|---|
| | Component | 26 | 27 | 28 | 29 | 30 | 31 |
| Active ingredient | Fentanyl | 10 | 12 | 15 | 10 | 12 | 15 |
| Acrylate-rubber | Duro-Tak ™ 87-502B | 85 | 86 | 80 | | | |
| hybrid adhesive | Duro-Tak ™ 87-504B | | | | 85 | 86 | 80 |
| Absorption | Brij ™ 30 | 5 | | | 5 | | |
| enhancer | Plurol oleique ™ CC497 | | 2 | | | 2 | |
| | Labrafil ™ | | | 5 | | | 5 |

| | | | | | | |
|---|---|---|---|---|---|---|
| Matrix Thickness (μm) | 40 | 50 | 60 | 40 | 50 | 60 |

### Comparison of Hybrid Adhesives

The acrylate-rubber hybrid adhesives classified three different types (Table 1-2) according to the presence of a cross-liking agent and a tackifier. Also, it can be distinguished by two groups of solvent system (Table 1-3). The compositions of two solvent systems [Group A (502A, 503A and 504B) & Group B (502B and 504B)] are described in Table 1-3. During the formulation development, the solid part of adhesive is solved in the solvents, in which the drug substance and other excipients can be dissolved in.

Therefore, even though the chemical structure of adhesive is the same, but the formulation for developing a transdermal patch should be modified significantly according to the solvent compositions. Since their physical properties and the compatibility of adhesives to drug substance were changed, their formulation development of patch should be approached with totally different methods to maintain the better stability of the final formula.

TABLE 6
Types of Hybrid Pressure Sensitive Adhesive (PSA)

| | | Functional | Cross linker |
|---|---|---|---|
| PSA | Chemical composition | group | added |
| 87-502A | Acrylic-rubber hybrid | —OH | X |
| 87-502B | | | |
| 87-503A | Acrylic-rubber hybrid | | ◯ |
| 87-504A | Acrylic-rubber hybrid tackifier | | ◯ |
| 87-504B | | | |

TABLE 7
Solvent System of Hybrid PSA

| PSA | SOLVENT (%) |
|---|---|
| 87-502A, 87-503A, 87-504A | Ethyl acetate: 45 |
| | n-heptane: 31 |
| | n-hexane: 24 |
| 87-502B | Ethyl acetate: 30-60 |
| | n-heptane: 10-30 |
| 87-504B | Ethyl acetate: 30-60 |
| | n-heptane: 10-30 |
| | Acetylacetone: 0.1-1 |

### Comparative Examples 1 to 4

Following the procedure disclosed in Korean Patent Publication 10-2009-0101579, the contents of which is incorporated herein by reference, comparative examples of the transdermal drug delivery systems were prepared using acrylate adhesive having hydroxyl functional group (Duro-Tak™ 87-2510) and absorption enhancer selected from Brij™ 30, Plurol Oleique™ CC497, or Lauroglycol™ and by the same method described in Example 1-31. The detailed composition is provided in Table 8. In addition, the commercially available Durogesic™ D-trans (manufacturer: Janssen Korea) was used as the Comparative Example 4.

TABLE 8

| | | Comparative Example | | |
|---|---|---|---|---|
| | | (% by weight) | | |
| | Component | 1 | 2 | 3 |
| Active ingredient | Fentanyl | 10 | 10 | 10 |
| Acrylate-rubber hybrid | Duro-Tak ™ 87-2510 | 85 | 88 | 85 |
| adhesive | | | | |
| Absorption enhancer | Brij ™ 30 | 5 | | |
| | Plurol oleique ™ CC497 | | 2 | |
| | Lauroglycol ™ | | | 5 |

| | | | |
|---|---|---|---|
| Matrix Thickness (μm) | 40 | 40 | 40 |

### Experimental Example 1

### Measurement of Skin Penetration Rate of the Transdermal Drug Delivery Compositions According to Adhesives

The transdermal drug delivery systems prepared in Example 1-31 and Comparative Examples 1 to 4 were applied onto hairless mouse skins and Human cadaver skin (58 year old male or 68 year old female), for determining their skin penetration rates. Specifically, skins were excised from hairless mice (6 to 8 weeks old) right before the experiment. Each transdermal drug delivery system was cut in a circular form having a size of 2 cm² and then attached to the isolated skins Each resulting skin was fixed in each flow-through diffusion cell with a clamp thereof. To the receiver thereof, was added an isotonic phosphate buffer solution (pH 6.0). While the diffusion cell was maintained at 37° C. under stirring with a magnetic stirrer, samples were collected at an interval of 4 hours for 24 hours. The samples were subject to quantitative analysis using high-performance liquid chromatography under the following conditions as provided in Table 9 and the results are presented in Table 10.

TABLE 9

| | |
|---|---|
| Column | C-18 (Shiseido, 4 × 150 mm cm, 5 μm) |
| Mobile phase | Acetonitrile/water/phosphate buffer/triethanol amine |
| | (155/310/0.5/0.5) |
| Flow rate | 1 mL/min |
| Wavelength | 210 nm |
| Temperature | 30° C. |

TABLE 10
Penetration rate measured via hairless mouse skin

| | flux (μg/cm²/h) |
|---|---|
| Example 1 | 10.41 |
| Example 2 | 15.1 |
| Example 3 | 12.2 |
| Example 4 | 12.8 |
| Example 5 | 12.5 |
| Example 6 | 13 |
| Example 7 | 14.5 |
| Example 8 | 20 |
| Example 9 | 14.9 |
| Example 10 | 14.8 |
| Example 11 | 17 |
| Example 12 | 10.6 |
| Example 13 | 9.8 |
| Example 14 | 16.6 |
| Example 15 | 15.3 |
| Example 16 | 14.9 |
| Example 17 | 12.8 |
| Example 18 | 13.2 |
| Example 19 | 12.9 |
| Example 23 | 10.34 |
| Example 24 | 9.71 |
| Example 25 | 10.35 |
| Comparative Example 1 | 5.18 |
| Comparative Example 2 | 5.35 |
| Comparative Example 3 | 4.63 |
| Comparative Example 4 | 7.7 |

TABLE 11
Penetration rate measured via human cadaver skin

| | flux (μg/cm²/h) |
|---|---|
| Example 20 | 4.1 |
| Example 21 | 5.0 |
| Example 22 | 4.5 |
| Comparative Example 4 | 2.19 |

From the results shown in Tables 10-11, it can be seen that the transdermal drug delivery systems according to the present invention showed higher skin penetration rate than those of Comparative Examples 1-4.

### Experimental Example 2

### Measurement of Stability in an Accelerated Condition

In an accelerated stability test (40° C./75% RH) for 4 weeks, all tested formulations showed to be stable measured by content assay showing <0.3% of impurities.

TABLE 12
Stability Test Result at accelerated condition (40° C./75% RH, N = 6)

| | Week 0 | | Week 1 | | Week 2 | | Week 4 | |
|---|---|---|---|---|---|---|---|---|
| | Assay | | Assay | | Assay | | Assay | |
| | Impurity | | Impurity | | Impurity | | Impurity | |
| Example 23 | 97.5% | None | 102.2% | 0.2%* | 99.4% | 0.12%* | 98.8% | 0.10% |
| Example 24 | 103.6% | None | — | — | — | — | 98.5% | None |
| Example 25 | 98.6% | None | 98.5% | 0.2%* | 97.9% | 0.25% | 98.0% | 0.25% |

(Note: *one sample out of six samples)

Among the stable drug delivery system, those prepared using acryl-rubber hybrid adhesive 87-502B or 87-504B presented a better stability than those with 87-502A or 87-504A, respectively.

We claim:

1. A transdermal drug delivery system comprising:
(a) fentanyl or a pharmaceutically acceptable salt thereof as an active ingredient; and
(b) an acrylate-rubber hybrid adhesive,
wherein, the acrylate-rubber hybrid adhesive is prepared by a process without n-hexane.

2. The transdermal drug delivery system of claim 1, wherein the transdermal drug delivery system consists of a backing layer, the drug-containing matrix layer, and a release layer.

3. The transdermal drug delivery system of claim 1, wherein the acrylate-rubber hybrid is an acrylic polymer comprising a C₄-C₁₈ alkyl acrylate monomer grafted with a rubber macromer having a glass transition temperature of not more than −30° C.

4. The transdermal drug delivery system of claim 1, wherein the fentanyl or its pharmaceutically acceptable salt is present in an amount ranging from 5 to 40% by weight, based on the total weight of the drug-containing matrix layer.

5. The transdermal drug delivery system of claim 1, wherein the acrylate-rubber hybrid is present in an amount ranging from 60 to 95% by weight, based on the total weight of the drug-containing matrix layer.

6. The transdermal drug delivery system of claim 1, further comprising an acrylate polymer or a methacrylate polymer as a crystallization-inhibiting agent.

7. The transdermal drug delivery system of claim 6, wherein the crystallization-inhibiting agent is present in an amount ranging from 1 to 10% by weight, based on the total weight of the drug-containing matrix layer.

8. The transdermal drug delivery system of claim 6, wherein the crystallization-inhibiting agent is a copolymer of butyl methacrylate, 2-dimethylaminoethyl methacrylate, and methyl methacrylate in a weight ratio of 1:2:1.

9. The transdermal drug delivery system of claim 1, further comprising one or more absorption enhancers selected from the group consisting of terpenes; surfactants; polyoxyethylene alkyl ethers; fatty alcohols; sugar esters; glycerols; alkyl 2-ethyl hexanates; and diethoxylethyl succinates.

10. The transdermal drug delivery system of claim 9, wherein the absorption enhancer is present in an amount ranging from 1 to 20% by weight, based on the total weight of the drug-containing matrix layer.

11. The transdermal drug delivery system of claim 9, wherein the absorption enhancer is one or more selected from the group consisting of polyethylene glycol palm kernel glyceride, polyoxyethylene lauryl ether, polyglyceryl-3 oleate, lauryl alcohol, and oleyl alcohol.

12. The transdermal drug delivery system of claim 9, wherein the acrylate-rubber hybrid adhesive is prepared using ethyl acetate and n-haptane.

13. The transdermal drug delivery system of claim 9, wherein the acrylate-rubber hybrid adhesive is Duro-Tak™ 87-502B or Duro-Tak™ 87-504B.

14. A method of preparing the transdermal drug delivery system of claim 1 comprising:
(i) mixing fentanyl with an acrylate-rubber hybrid adhesive;
(ii) adding a solvent to the mixture obtained from step (i);
(iii) casting the solution obtained from step (ii) on a release liner coated with silicone;
(iv) drying the casted mixture obtained from (iii);
(v) laminating a polyethylene film onto the dried layer obtained from step (iv) to form a backing membrane to prepare a transdermal drug delivery system.
